(19) United States
(12) Patent Application Publication
(10) Pub. No.: US 2020/0087699 A1
(43) Pub. Date: Mar. 19, 2020

(54) METHODS FOR PRODUCING ZOOPLANKTON

(71) Applicant: WATERFORD INSTITUTE OF TECHNOLOGY, Waterford (IE)

(72) Inventors: Rafael HERENA GARCIA, Waterford (IE); John NOLAN, Waterford (IE); Alfonso PRADO-CABRERO, Waterford (IE)

(21) Appl. No.: 16/469,959

(22) PCT Filed: Dec. 12, 2017
(86) PCT No.: PCT/EP2017/082490

§ 371 Date: Jun. 14, 2019

(30) Foreign Application Priority Data
Dec. 15, 2016 (GB) 1621386.0

(51) Int. Cl. C12P 23/00; C12N 1/12; A23L 33/105; A61K 8/9706

(52) U.S. Cl. CPC C12P 23/00; A61K 8/9706; A23L 33/105; C12N 1/12

(57) ABSTRACT

A method of producing zooplankton biomass rich in a target compound, the method comprising the steps of: (a) providing one or more species of microalgae and/or cyanobacteria; (b) optionally stimulating the microalgae and/or cyanobacteria; (c) contacting the microalgae and/or cyanobacteria with one or more species of zooplankton which feed thereon; and (d) collecting a portion of the zooplankton; wherein waste from the zooplankton is fed back to the microalgae and/or cyanobacteria

The present invention relates to a process aimed to obtain natural metabolites with commercial interest from microalgae and/or cyanobacteria and/or zooplankton species at large scales. The invention suitably enables the continuous production and exploitation of these metabolites, especially carotenoids, for example lutein or astaxanthin, on a large scale in a cost effective and environmentally friendly manner.

Due to its high nutritional value zooplankton is typically used in aquaculture to rear fish larvae, a fish development stage where highly nutritious living feed are important in the production of adult fish. Consequently, the main consumers of zooplankton are hatcheries, and previous attempts of setting up zooplankton mass cultures have been performed for this purpose. Microalgae are commercially cultured to produce carotenoids (for example β-carotene and astaxanthin) or high value fatty acids (for example DHA, docosahexaenoic acid and EPA, eicosapentaenoic acid). Although generally considered the best option to feed zooplankton, microalgae are only partially used in hatcheries for zooplankton culture due to the high costs associated to its own production. Cheaper non-algae meals (for example micronized rice bran, yeast, etc.) are thus often used to produce zooplankton in aquaculture, despite the fact that the quality of the zooplankton produced is lower.

Cost has thus been considered a drawback of producing zooplankton at large scale using algae as feed. However some attempts have been made to do this. Sorgeloos et al. (Tobias, W. J., P. Sorgeloos, E. Bossuyt and O. A. Roels (1979). “The Technical Feasibility of Mass-Culturing Artemia Salina in the St. Croix “Artificial Upwelling” Mariculture System.” Proceedings of the World Mariculture Society 10(1-4): 203-214), used an open flow-through system where algae were obtained from coastal upwellings. High productivity was achieved (8.7 Kg of zooplankton per m³ tank in 14 days), but the system was totally dependent on the availability of natural coastal upwellings rich in microalgae.

The potential of algae for the production of high value products is largely known, but zooplankton has not been extensively researched as source of valuable compounds, despite the fact that many species accumulate carotenoids such as astaxanthin or polyunsaturated fatty acids such as EPA or DHA. In the present invention the combination of both organisms can add value to the products that can be obtained from them separately, as zooplankton can transform low value carotenoids or fatty acids from algae into high value carotenoids or fatty acids. More importantly, processing zooplankton biomass for product extraction is more feasible than processing microalgae, because zooplankton has a higher size that makes its collection and dewatering easier when compared to microalgae. The main limitation to the development of this strategy is the difficulty of producing enough algae to culture enough zooplankton in order to obtain profitable amounts of the compounds of interest. The only current examples of the combined use of microalgae and zooplankton are krill and Calanus finmarchicus, whose extracted oils are rich in DHA, EPA and contains low concentrations of astaxanthin. However, krill and Calanus are not currently cultured, but captured in the sea.

The first attempts of culturing zooplankton with microalgae as feed had as its objective to study diverse aspects of the physiology, growth rate and population dynamics of the zooplankton in relation with the algae used. These laboratory experiments used dual-stage chemostats where the first stage was occupied by the algae and the second one by the zooplankton. In a chemostat, the organism studied grows at steady state, due to the continuous provision of new media and the continuous extraction of old media containing the organism. Of note, the new media provided contains one of the nutrients at a concentration lower than needed by the organism, causing its growth to be constant, but at a rate below its maximum growth rate due to the low concentrations of the nutrient chosen as limitant.

The turbidostat principle has also been used to study zooplankton population dynamics in the laboratory using microalgae as feed, but this time, as per the turbidostat principle of work, with the zooplankton species growing near its maximum growth rate. In this process the algae concentrations is monitored by a turbidimeter where the zooplankton is growing. When the turbidometry decays, new algae are injected to the turbidostat, thus maintaining the zooplankton growth rate close to its maximum.

The above described two-stage reactors are open processes, that is, water is provided at the start and discarded at the end of the process, with the addition of new water (including nutrients), being necessary to keep the process working.

Recirculating aquaculture systems (RAS) are used to produce fish in a controlled environment, in which the discharge of water into the environment is minimized and the water is treated and re-circularized to be used again in the pools where the fish is cultured. This farming system typically relies on a series of mechanical, physical or chemical devices to purify water, with only bacteria as living organisms with a role in purifying water to convert ammonium into less toxic nitrate. Physical means to purify the water include but are not limited to solids collection systems, filters to remove nitrate from water produced by bacteria, protein skimmers to remove dissolved organic matter, water sterilization treatments such as UV or ozonation to eliminate organisms that can cause disease outbreak in the fish, pH control systems and systems for aeration or oxygenation of water. RAS can be combined with plants to remove ammonia and nitrate from the water, a system thus known as aquaponics. RAS has been adapted and applied to the production of the zooplankton species Brachionus plicatilis using the microalgae Tetraselmis suecica (Sananurak 2009) at pilot scale.

Integrated multi-trophic Aquaculture system (IMTA) is another approach to aquaculture where, instead of using mainly physical devices to purify water like in RAS, living organisms are used. With economic feasibility as aim, these purifying organisms are chosen from species that can be commercialized. Therefore, instead of a biofilter and a nitrate filter as used in RAS, typically macroalgae are incorporated to IMTA in order to fulfil these two nitrogen purification steps, and instead of solid waste collectors, typically sea cucumbers and shellfish are used. IMTA can be run in the sea (marine-IMTA), where the species cited are confined in cages and marine currents distribute the wastes of each organism to be used by other organisms in the IMTA. The economic feasibility of implanting IMTA on land (land-based IMTA) is also being studied, even using recirculation systems. This last type of IMTA pursues the zero-water exchange with the environment, and an example of this type of IMTA is being researched at Florida Atlantic University. Zooplankton has been proposed to be part of IMTA to assist clean solid waste from fish and obtain in exchange a biomass rich in unsaturated fatty acids.

Carotenoids are amongst the most interesting and valuable molecules produced by algae. Furthermore, some of these carotenoids can be converted into more valuable carotenoids when assimilated by zooplankton. A number of the carotenoids produced by microalgae, namely lutein, zeaxanthin and β-carotene, are essential for human life. Others, like astaxanthin, which is produced by Haematococcus and many zooplankton species, have shown to be beneficial for human health. In particular, lutein and zeaxanthin have been related with a lower risk of developing the later stage of age-related macular degeneration (AMD) in subjects with low carotenoid consumption, and the addition of meso-zeaxanthin to the two former carotenoids, has been demonstrated to improve vision in terms of contrast sensitivity in healthy individuals. In addition, it is believed that the macular carotenoids may have a role in cognitive function and Alzheimer's disease. Astaxanthin is a keto-carotenoid, and is best known for its use in aquaculture and poultry, but like lutein, zeaxanthin and meso-zeaxanthin, it is also used to prepare health supplements for human consumption, due to the growing body of evidence supporting the protective role of this carotenoid against oxidative stress and inflammation.

Microalgae are also exploited to obtain high value fatty acids for example DHA and EPA. It has also been reported that these fatty acids can be produced by certain zooplankton species from shorter fatty acids obtained from the diet. DHA is a main component of the brain, the retina and the heart, and is mainly produced to fortify infant formula and for the food, beverage and health supplement industries. EPA acts as an eicosanoid, regulating inflammation and immunity in humans. Its commercial production from microalgae only recently started, with the objective of supplying the health supplement, pharmaceutical and aquaculture markets. Microalgae have also attracted interest for the production of biofuels, making use of their saturated fatty acids to obtain it. However, the costs associated with algae culturing and processing have prevented profitable commercialization of this process to date. Other lipid compounds that can potentially be obtained from microalgae include phytosterols, sterol like molecules which are of increasing commercial interest due to their ability to lower cholesterol levels in blood. Furthermore, these compounds have been reported to be involved in anti-inflammatory, anti-atherogenicity, anti-cancer and anti-oxidative activities.

Recently, tools to introduce foreign genes on the zooplankton species Artemia sp. have been developed. This involves using Artemia in a bioreactor to produce a protein of interest, for example eukaryotic therapeutic proteins such as growth hormone, insulin or monoclonal antibodies. Although this technology is in its infancy, there is significant potential when compared with other protein production systems, such as Chinese hamster ovary (CHO) cells or bacteria. Whilst the former are used to produce complex proteins with the adequate posttranslational modifications needed for their activity, the scalability of this system is not cost-effective. On the other hand, bacteria used for exogenous protein production produce higher yields, but they only can produce simple proteins. Zooplankton species have the potential to overcome the limitations of both production systems, as their position in evolution allows them to produce complex proteins, and furthermore, the opportunity of growing this zooplankton at high scale would allow for the production of high amounts of the protein of interest.

The present invention provides a continuous process that facilitates extraction of useful compounds from an algae/zooplankton system.

According to a first aspect of the present invention there is provided a method of producing zooplankton biomass rich in a target compound, the method comprising the steps of:

(a) providing one or more species of microalgae and/or cyanobacteria;

(b) optionally stimulating the microalgae and/or cyanobacteria;

(c) contacting the microalgae and/or cyanobacteria with one or more species of zooplankton which feed thereon; and

(d) collecting a portion of the zooplankton;

wherein waste from the zooplankton is fed back to the microalgae and/or cyanobacteria.

Suitably in step (a) the microalgae and/or cyanobacteria are provided in a first chamber. After optional stimulation in step (b), they are contacted with zooplankton in step (c), suitably in a second chamber.

Thus the first aspect of the present invention suitably provided a method of producing zooplankton biomass rich in a target compound, the method comprising the steps of:

(a) providing one or more species of microalgae and/or cyanobacteria in a first chamber;

(b) optionally stimulating the microalgae and/or cyanobacteria;

(c) contacting the microalgae and/or cyanobacteria with one or more species of zooplankton which feed thereon in a second chamber; and

(d) collecting a portion of the zooplankton;

wherein waste from the zooplankton is fed back to the microalgae and/or cyanobacteria.

Any microalgae or cyanobacteria species which is able to synthesise a useful target compound may be used in the present invention. Suitably species include those belonging to the genera Dunaliella, Rhodomonas, Chroomonas, Tetraselmis, Chlorella, Scenedesmus, Nannochloropsis, Isochrysis, Sticochrysis, Monochrysis, Pavlova, Chaetoceros, Thalassiosira, Muriellopsis, Botryococcus, Phaeodactylum, Platymonas, Haematococcus, Spirulina and Galdieria. Some preferred species for use herein include Dunaliella salina, Haematococcus pluvialis, Chlorella spp., Scenedesmus spp., Muriellopsis spp., Chlorella spp., Dunaliella viridis, Chlorella ellipsoidea, Dunaliella salina, Dunaliella spp., Botryococcus braunii, Phaeodactylum tricornutum, Spirulina platensis and Galdieria sulphuraria. Preferably the present invention uses one or more species of microalgae.

Any species of zooplankton that feeds on the microalgae and/or cyanobacteria may be used in the present invention. Preferred species of zooplankton belong to the phylum Rotifera, to the subclass Copepoda or to the orders Cladocera, Euphausiacea, Mysida, Anostraca including the families Artemiidae, Branchinectidae, Branchipodidae, Chirocephalidae, Parartemiidae, Streptocephalidae, Tanymastigidae and Thamnocephalidae.

Although the zooplankton used in the invention feed on microalgae and/or cyanobacteria, they may also feed on bacteria, archaea, fungi, other zooplankton, protozoa, fecal pellets, detritus, or any other type of decaying living matter present in the system.

In a second aspect the present invention suitably provides a method of providing a composition comprising a target compound, the method comprising producing a zooplankton biomass rich in the target compound according to steps (a) to (d) of the method of the first aspect, and which further involves a step (e) of extracting the target compound from the zooplankton.

According to a third aspect of the present invention there is provided a system for producing biomass rich in a target compound, the system comprising:
(i) a first chamber comprising microalgae and/or cyanobacteria;
(ii) a second chamber comprising zooplankton;
(iii) means for carrying the microalgae and/or cyanobacteria from the first chamber to the second chamber;
(iv) means for carrying waste from the second chamber to the first chamber;
(v) means for collecting a portion of the zooplankton; and optionally
(vi) means for stimulating the microalgae in the first chamber.

Preferred features of the second and third aspects are as defined in relation the first aspect. Further preferred features of the first, second and third aspects will now be described.

The first chamber, where microalgae and/or cyanobacteria are cultured and may be stimulated, is connected to the second chamber, where zooplankton is cultured. Suitably there is a conduit which carries the composition comprising the microalgae and/or cyanobacteria from the first chamber to the second chamber. The conduit, may for example, be an overflow pipe. In order to close the system, the second chamber is connected to the first chamber by a different pipe. This connection is suitably free of any non-biological water treatment system.

Suitably the system of the present invention includes a filter configured to keep zooplankton in the second chamber but allow microalgae and/or cyanobacteria to pass into the second chamber.

The invention involves zooplankton collection. This may be achieved by any suitable means. Suitably a percentage of the zooplankton is periodically collected by filtration in order to ensure maximum constant zooplankton production. Suitable filtration equipment will be known to the person skilled in the art. The collected zooplankton may be optionally dried for preservation before extracting the target compound.

Suitably the present invention involves a multi trophic cycle (i.e. bacteria, archaea, fungi, microalgae, cyanobacteria, crustacean) long term, where the microalgae and/or cyanobacteria population, via photosynthesis, adds biomass continuously to the system. This biomass, which may contain the target compound(s) synthesized by the microalgae and/or cyanobacteria, is continuously provided to the zooplankton in order to promote its growth and its enrichment with the target compound(s). The zooplankton can at the same time further metabolize a number of low value compounds provided by the microalgae, converting them into added value compounds (for example β-carotene into astaxanthin). Finally, the zooplankton biomass, containing the target compound(s), is harvested in order to extract such compounds. The process is continuous and involves a substantially closed loop, as the wastes produced by the zooplankton are transferred back to the microalgae to support its growth.

The microalgae and/or cyanobacteria and the zooplankton are provided in an aqueous media that may comprise other nutrients and/or microorganisms.

Zooplankton may secrete nitrogen to the media, mainly in the form of ammonia. This may be poisonous at even low concentrations. Suitably autotrophic nitrifying bacteria and archaea are present which oxidise ammonia into nitrite, and other bacteria species further oxidizes this nitrite to nitrate. Nitrate is far less poisonous and readily usable for microalgae to grow. The joint action of these two types of microorganisms is known as nitrification. These groups of bacteria may be allowed to naturally colonize the system within a few weeks, or alternatively these bacteria can be acquired commercially and added to the system, thus accelerating the process. These bacteria and archaea are aerobic, meaning that they need oxygen to grow and perform nitrification. In the present invention oxygen is provided by microalgae.

Zooplankton may also excrete carbon dioxide, which establishes a reaction equilibrium with water to form carbonic acid, a molecule that dissociates to liberate a proton that contributes to water acidification. Microalgae uptakes carbon dioxide during the day and secretes it during the night, decreasing and increasing pH during these two periods respectively and buffering the effect of the carbon dioxide produced by the zooplankton. The net result of this is a stable equilibrium of pH within the system.

Zooplankton may excrete faeces and produce carapaces after entering new developmental stages. Dead individuals and decaying microalgae and bacteria are also gradually deposited at the bottom of the zooplankton tank. These solid materials altogether form a sludge on the bottom of the zooplankton phase. This solid material has to be cleared in order to prevent its build-up. This can be achieved periodically by mechanical means. Alternatively, this sludge can be decomposed within the reactor to liberate captured nitrogen, phosphorus, metals, etc. for reuse of microalgae and zooplankton growth. In order to treat this sludge, specialised bacteria, archaea and/or fungal strains may be provided which colonise gradually the sludge. These microorganisms can be acquired commercially. In order to accelerate the decomposition of the sludge, the bacteria can be assisted by added protozoa and other species of zooplankton like rotifers, thus converting the sludge in activated sludge. As the protozoa and the added zooplankton organisms feed on the sludge, they break-up the solid materials deposited, thus facilitating the access of bacteria to this material for its final decomposition and liberation to the aqueous media of the nutrients enumerated above. Oxygen supply is important in this process, and it is provided by the photosynthetic activity of microalgae in the system. Suitably the carbon to nitrogen ratio is maintained low, in order to avoid bacterial overgrowth that can capture and use nitrate and phosphate essential for algal growth.

Alternatively, if the carbon to nitrogen ratio is high, heterotrophic bacterial growth is promoted, thus converting the activated sludge into bioflocs, which can serve as additional feed source for the zooplankton if the zooplankton's feeding behaviour allows it to benefit from them. It is important that the bioflocs and microalgae populations are balanced, in order to secure enough microalgae biomass in the system to provide the appropriate oxygen supply for bacterial ammonia oxidation. Also, the filtration equipment must be able to afford the presence of these particles in the system in order to keep both phases effectively communicated by water flow. In some embodiments bacteria producers of carotenoids may be introduced in the system, in order to serve as a source of these molecules complementary to microalgae.

In order to preserve the integrity of the algae phase, it should be kept free of grazing zooplankton or protozoa. Suitably the algae phase may be provided with a device that mechanically kills or damage zooplankton and protozoa escaping the filter or coming from the environment. This device must be innocuous for the microalgae. The algae phase can thus provide microalgae indefinitely to the system.

The present invention suitably relates to a closed loop two-phase bioreactor, where two different aquatic organisms are cultured inside in two separate chambers. The waste of one of these two organisms may be converted to nutrients by a coexisting population of at least bacteria and optionally archaea and fungi. The configuration of the reactor aims to encourage the food chain processes between two trophic levels represented by the two organisms cultured, in order to maximise biomass production and promote the system's self-control. The first species is the feed of the second, and the waste products of the second are converted into newly available nutrients (mainly phosphorus, nitrogen and vitamins), which are the essential components for growing of the first species.

The main advantages of a closed loop process of the invention that (i) only minimal amounts of of new water (typically less than 1% vol) need to be added daily, (ii) a percentage of nutrients needed by algae (nitrogen, phosphorus and vitamins) are obtained from the zooplankton waste due to bacteria activity, and (iii) pH basification of the media by algae is compensated by pH acidification by zooplankton. Moreover, the processing of the zooplankton biomass in order to extract the compounds of interest is more feasible than processing microalgae biomass. The small amounts of water added are to replace loss through evaporation of water used by the organisms (e.g. in photosynthesis).

Suitably the method of the first and second aspects of the present invention involves a closed loop process.

Suitably the method of the present invention is a continuous process. Suitably the method involves only minimal addition of water to the system, and suitably no water is removed.

In certain embodiments, the present invention provides an alternative means of producing and exploiting carotenoids on a large scale which is less expensive, reliable and has environmental benefits. Microalgae and cyanobacteria are beneficial sources of carotenoids because they have fast growth rates, do not require much space and can be grown in bioreactors on land that is not suitable for agriculture. The method described herein involves the use of microalgae and/or cyanobacteria to culture zooplankton, which in turn will become enriched in the carotenoids produced by the microalgae. Final collection and extraction of zooplankton biomass is advantageous in terms of feasibility when compared to microalgae.

The first step of the present invention involves growing microalgae and/or cyanobacteria biomass, preferably comprising the target compound or a precursor of the target compound that may be converted to the target compound by zooplankton. In some embodiments the microalgae may produce the target compound or its precursor during its normal life cycle activities. However the production of the target compound or precursor can be stimulated and/or enhanced by changing the conditions in which the microalgae lives and reproduces and grows.

Step (b) of the method of the present invention is an optional step. When present step (b) involves stimulating the microalgae and/or cyanobacteria. Suitably step (b) involves applying a stress to the microalgae and/or cyanobacteria. For example step (b) may involve irradiation with a specific light quality or nutrient deprivation.

In step (c) the microalgae and/or cyanobacteria are transferred to the second chamber to feed the zooplankton, and the target compound or precursor consumed can be stored in the lipid depots and other parts of the animal (e.g. and muscle). Depending on the size of the zooplankton species used, the partially digested microalgae present in its digestive apparatus can significantly contribute to the total target compound content. In some embodiments the zooplankton species holds the enzymatic machinery to introduce modifications in the target compound precursors sourced from the microalgae and/or cyanobacteria. For example this may involve converting either β-carotene or zeaxanthin from the microalgae into canthaxanthin or astaxanthin. Therefore, the zooplankton can contain one or more than one target compound, for example one or more than one carotenoid.

In step (e) of the method of the present invention, the carotenoid compounds from the zooplankton, the final extracted composition may comprise one or more than one target compound, for example one or more than one carotenoid.

Extraction is typically carried out using physical and/or chemical means known to the person skilled in the art.

In some preferred embodiments the target compound is a carotenoid. Suitable carotenoids that may be obtained according to the method of the present invention include β-carotene, astaxanthin, canthaxanthin, lutein, zeaxanthin, phytoene, phytofluene, echinenone and fucoxanthin.

In some embodiments, the present invention uses certain species of microalgae and zooplankton to produce DHA and EPA, alone, in combination, in combination with other fatty acids and/or in combination with one or more carotenoids. Zooplankton species accumulate fatty acids sourced from their diet. If the microorganisms used as feed contain high value fatty acids (e. g. DHA and EPA), the zooplankton can store them in their lipid depots. Alternatively, certain species of zooplankton hold dedicated enzymes to further metabolise the fatty acids ingested in order to increase the number of carbons and desaturations of the backbone of the fatty acid. In some preferred embodiments, the zooplankton species used can convert low value fatty acids into high value fatty acids like DHA and/or EPA. Furthermore, phytosterols produced by microalgae could be stored and accumulated by zooplankton, making it a potential source of these molecules.

In some preferred embodiments, carotenoids may be extracted within a matrix of polyunsaturated fatty acids. Fatty acids with commercial interest such as linoleic acid (LA), α-linolenic acid (ALA), γ-linolenic acid (GLA), arachidonic acid (ARA), docosahexaenoic acid (DHA), eicosapentaenoic acid (EPA), may be produced as a carotenoid co-product, adding value. In this specification we may refer to a product containing one or more carotenoids and one or more polyunsaturated fatty acids as carotenoids rich oil. The product obtained by the method of the present invention is suitably a composition comprising one or more natural pigments, preferably one or more carotenoid compounds. Suitably the composition includes one or more further compounds. Preferably the composition is an oil rich composition. In highly advantageous embodiments the present invention provides oil which is rich in carotenoids.

Thus the second aspect of the present invention may suitably provide a method of producing an oil rich in a target compound, the method comprising the steps of:
(a) providing one or more species of microalgae and/or cyanobacteria;
(b) optionally stimulating the microalgae and/or cyanobacteria;
(c) contacting the microalgae and/or cyanobacteria with one or more species of zooplankton which feed thereon;
(d) collecting a portion of the zooplankton; and
(e) extracting an oil from the zooplankton;

wherein waste from the zooplankton is fed back to the microalgae and/or cyanobacteria.

In some embodiments, the present invention may use certain species of microalgae and/or cyanobacteria and zooplankton to produce exogenous proteins. Suitably the target compound is an exogenous protein. The tools needed to introduce exogenous genes and enable them to produce a protein of interest have been described in Artemia sp. The present invention suitably provides a process for the continuous production of zooplankton species, which means that it can be applied to the continuous, long term and constant yield production of Artemia or any other zooplankton species that can be genetically engineered in order to obtain high quantities of a protein of interest.

The first aspect of the present invention relates to a method of producing biomass rich in a target compound.

The second aspect provides a composition comprising the target compound.

Preferably the composition directly from the method of the second aspect of the present invention is safe for human consumption.

According to a fourth aspect of the present invention there is provided a composition obtained by the method of the second aspect.

The composition of fourth aspect may be used as a food supplement or nutraceutical composition.

The present invention may also provide the use of a composition of the fourth aspect in a cosmetic composition.

The present invention may also provide the use of a composition of the fourth aspect as an antioxidant.

The present invention may also provide the composition of the fourth aspect for use as a medicament. Such a medicament may be used for prophylactic or therapeutic treatment.

The present invention relates to a process involving a closed loop including two phases where organisms belonging to different trophic levels are cultured (suitably microalgae and/or cyanobacteria and zooplankton), microalgae serving as feed for the zooplankton and wherein wastes of the zooplankton are converted by bacterial activity into nutrients (e.g. nitrate, phosphate and vitamins). These nutrients are recirculated to support the growth of microalgae, which in turn serve again as feed for the zooplankton.

The present invention provides a methodology of producing at large scales biomass rich in a compound or metabolite with commercial interest.

In some embodiments microalgae may provide lutein to the zooplankton where it is stored and extracted.

In some embodiments microalgae may provide precursor carotenoids such as β-carotene or zeaxanthin to the zooplankton where it is metabolized into astaxanthin, stored and extracted.

In some embodiments microalgae may provide lutein to the zooplankton where it is stored, and may provide carotenoid precursors such as β-carotene and/or zeaxanthin to the zooplankton to produce astaxanthin, were it is stored and extracted together with lutein.

In some embodiments microalgae may provide lutein to the zooplankton where it is stored and extracted within a matrix that may contain high value fatty acids like linoleic acid (LA), α-linolenic acid (ALA), γ-linolenic acid (GLA), arachidonic acid (ARA), docosahexaenoic acid (DHA), or eicosapentaenoic acid (EPA).

In some embodiments microalgae may provide precursor carotenoids such as β-carotene or zeaxanthin to the zooplankton where it is metabolized into astaxanthin, stored and extracted within in a matrix that may contain high value fatty acids like linoleic acid (LA), α-linolenic acid (ALA), γ-linolenic acid (GLA), arachidonic acid (ARA), docosahexaenoic acid (DHA), or eicosapentaenoic acid (EPA).

In some embodiments microalgae may provide lutein to the zooplankton where it is stored and extracted and provides β-carotene and/or zeaxanthin to produce astaxanthin within in a matrix that may contain high value fatty acids like linoleic acid (LA), α-linolenic acid (ALA), γ-linolenic acid (GLA), arachidonic acid (ARA), docosahexaenoic acid (DHA), or eicosapentaenoic acid (EPA).

In some embodiments microalgae may provide biomass to the zooplankton where high value fatty acids like linoleic acid (LA), α-linolenic acid (ALA), γ-linolenic acid (GLA), arachidonic acid (ARA), docosahexaenoic acid (DHA), or eicosapentaenoic acid (EPA) is stored and extracted.

In some embodiments microalgae may provide biomass to the zooplankton where phytosterols, and carotenoids such as lutein and/or astaxanthin are stored and extracted within in a matrix that may contain high value fatty acids like linoleic acid (LA), α-linolenic acid (ALA), γ-linolenic acid (GLA), arachidonic acid (ARA), docosahexaenoic acid (DHA), or eicosapentaenoic acid (EPA).

In some embodiments microalgae may provide biomass to the zooplankton where exogenous proteins are synthesised, stored and extracted.

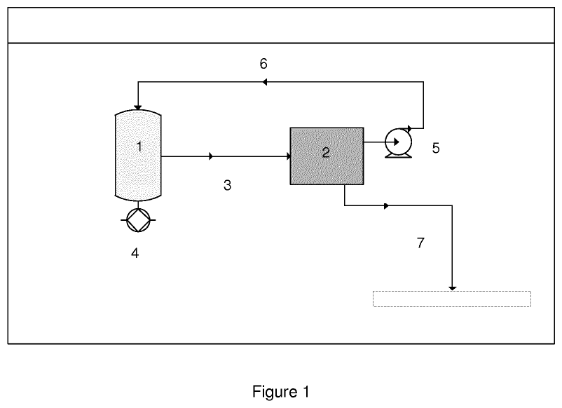

The invention may be further described with reference to FIG. 1. This flow diagram illustrates how the invention works in practice. Microalgae and/or cyanobacteria is provided in a first chamber 1 which is connected to a second chamber 2 comprising zooplankton. A conduit 3 allows algae to flow from chamber 1 to chamber 2. Aeration is provided by air pump 4. Waste water and nutrients from chamber 2 are directed by water pump 5 along channel 6 back to chamber 1. Zooplankton may be periodically harvested from chamber 2 via conduit 7.

1. A method of producing zooplankton biomass rich in a target compound, the method comprising the steps of:
(a) providing one or more species of microalgae and/or cyanobacteria;
(b) contacting the microalgae and/or cyanobacteria with one or more species of zooplankton which feed thereon; and
(c) collecting a portion of the zooplankton,
wherein waste from the zooplankton is fed back to the microalgae and/or cyanobacteria.

2. A method according to claim 1 wherein step (a) is carried out in a first chamber and step (b) is carried out in a second chamber.

3. A method of providing a composition comprising a target compound, the method comprising producing a zooplankton biomass rich in the target compound according to claim 1, and extracting the target compound from the zooplankton.

4. A system for producing biomass rich in a target compound, the system comprising:
(i) a first chamber comprising microalgae and/or cyanobacteria;
(ii) a second chamber comprising zooplankton;
(iii) means for carrying the microalgae and/or cyanobacteria from the first chamber to the second chamber;
(iv) means for carrying waste from the second chamber to the first chamber; and
(v) means for collecting a portion of the zooplankton.

5. The system of claim 4, wherein the system includes a closed loop.

6. The method of claim 1, wherein providing includes providing one or more species of microalgae.

7. The method of claim 1, wherein the target compound comprises lutein.

8. The method of claim 1, wherein the target compound comprises astaxanthin.

9. The method of claim 1, wherein the microalgae provides a precursor which is metabolised to produce the target compound by the zooplankton.

10. The method of claim 1, which produces an oil rich in one or more fatty acids selected from linoleic acid (LA), α-linolenic acid (ALA), γ-linolenic acid (GLA), arachidonic acid (ARA), docosahexaenoic acid (DHA), or eicosapentaenoic acid (EPA).

11. A composition obtained by the method of claim 3.

12. (canceled)

13. (canceled)

14. The method of claim 1, further comprising:
stimulating the microalgae and/or cyanobacteria between steps (a) and (b).

15. The system of claim 4, further comprising:
(vi) means for stimulating the microalgae in the first chamber.

16. The system of claim 4, wherein the target compound comprises lutein.

17. The system of claim 4, wherein the target compound comprises astaxanthin.

18. The system of claim 4, wherein the microalgae provides a precursor which is metabolised to produce the target compound by the zooplankton.

19. A medicament comprising the composition of claim 11.

20. A food supplement or nutraceutical comprising the composition of claim 11.

21. A cosmetic comprising the composition of claim 11.

22. An antioxidant comprising the composition of claim 11.
